(19) United States
(12) Patent Application Publication
(10) Pub. No.: US 2010/0069501 A1
(43) Pub. Date: Mar. 18, 2010

(54) Pharmaceutical Composition Comprising 6-Dimethylaminomethyl-1-(3-methoxy-phenyl)-cyclohexane-1,3-diol and Paracetamol

(75) Inventors: Petra Bloms-Funke, Wuerselen (DE); Klaus Schiene, Juechen (DE)

Correspondence Address: CROWELL & MORING LLP;INTELLECTUAL PROPERTY GROUP, P.O. BOX 14300, WASHINGTON, DC, 20044-4300, US

(73) Assignee: GRUENENTHAL GmbH, Aachen (DE)

(21) Appl. No.: 12/554,239

(22) Filed: Sep. 4, 2009

Related U.S. Application Data
(60) Provisional application No. 61/094,681, filed on Sep. 5, 2008.

(30) Foreign Application Priority Data
Sep. 5, 2008 (EP) 08 015 621.9

(51) Int. Cl. A61K 31/167; A61P 19/02

(52) U.S. Cl. 514/629

(57) ABSTRACT

A combination comprising (a) at least one 6-dimethylaminomethyl-1-(3-methoxy-phenyl)-cyclohexane-1,3-diol component, and (b) Paracetamol or a derivative thereof; a pharmaceutical composition and/or dosage form comprising such a combination as well as a method of treating one or more of pain and ostheoarthritis in a mammal characterized in that components (a) and (b) are administered simultaneously or sequentially to said mammal, wherein component (a) may be administered before or after component (b) and wherein components (a) or (b) are administered to the mammal either via the same or a different pathway of administration.

### CROSS REFERENCE TO RELATED APPLICATIONS

This application claims priority from co-pending U.S. provisional patent application No. 61/094,681, filed Sep. 5, 2008. Priority is also claimed based on European patent application no. EP 08 015 621.9, filed Sep. 5, 2009.

### BACKGROUND OF THE INVENTION

The present invention relates to a combination comprising (a) at least one 6-dimethylaminomethyl-1-(3-methoxy-phenyl)-cyclohexane-1,3-diol component, and (b) Paracetamol or a derivative thereof; a pharmaceutical combination and a dosage form comprising said combination as well as a method of treating one or more of pain and ostheoarthritis in a mammal characterized in that components (a) and (b) are administered simultaneously or sequentially to said mammal, wherein component (a) may be administered before or after component (b) and wherein components (a) or (b) are administered to the mammal either via the same or a different pathway of administration.

The treatment of pain conditions is extremely important in medicine. There is currently a worldwide demand for additional, not exclusively opioid-based, but highly effective pain treatment. The urgent need for action for patient-oriented and purposeful treatment of pain conditions, this being taken to mean the successful and satisfactory treatment of pain for the patient, is documented in the large number of scientific papers which have recently appeared in the field of applied analgesics and fundamental research work on nociception.

Even if the analgesics that are currently used for treating pain, for example opioids, NA- and 5HT-reuptake inhibitors, NSAIDS and COX inhibitors, are analgesically effective, side effects nevertheless sometimes occur. WO 2004/047823 describes substance combinations comprising certain analgesics including substituted 6-dimethylaminomethyl-1-phenyl-cyclohexane compounds and COX-II Inhibitors, which show super-additive effects upon administration. Due to the super-additive effect the overall dose and accordingly the risk of undesired side effects can be reduced.

### SUMMARY OF THE INVENTION

Thus, it was an object of the present invention to find further combinations that are suitable for the treatment of pain and which preferably exhibit fewer undesired side effects compared to its individual components, if administered in effective doses.

It has been found that a combination comprising (a) at least one 6-dimethylaminomethyl-1-(3-methoxy-phenyl)-cyclohexane-1,3-diol component, and (b) Paracetamol or a derivative thereof exhibits an analgesic effect. Said combination is also useful for the treatment of ostheoarthritis. If the components are present in the composition in such a weight ratio that a synergistic effect is observed after administration to the patient, the overall administered dose may be lowered, so that fewer undesired side-effects will occur.

Accordingly, the present invention relates to a combination comprising
(a) at least one 6-dimethylaminomethyl-1-(3-methoxy-phenyl)cyclohexane-1,3-diol component, and
(b) Paracetamol or a derivative thereof.

The 6-dimethylaminomethyl-1-(3-methoxy-phenyl)cyclohexane-1,3-diol component as used herein includes said compound in any possible form, thereby particularly including stereoisomers and salts. Also included are solvates and polymorphs of each of these forms.

Thus, in one embodiment the present invention relates to a combination comprising:
(a) 6-dimethylaminomethyl-1-(3-methoxy-phenyl)cyclohexane-1,3-diol of formula (I)

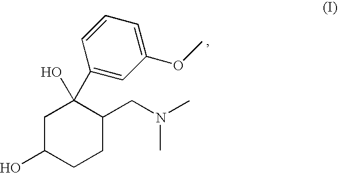

optionally in form of one of its stereoisomers, in particular an enantiomer or a diastereomer, a racemate or in form of a mixture of its stereoisomers, in particular enantiomers and/or diastereomers in any mixing ratio, or any corresponding salt thereof, and
(b) paracetamol or a derivative thereof.

In another embodiment the inventive combination comprises:
(a) (1RS,3RS,6RS)-6-dimethylaminomethyl-1-(3-methoxy-phenyl)-cyclohexane-1,3-diol of formula (I′),

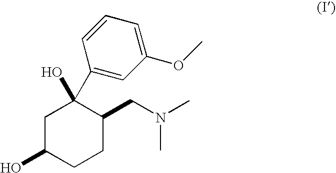

or a salt thereof, and
(b) paracetamol or a derivative thereof.

The (1RS,3RS,6RS)-6-dimethylaminomethyl-1-(3-methoxy-phenyl)-cyclohexane-1,3-diol stereoisomer of formula (I′) represents the racemate of the enantiomers (I′a) and (I′b):

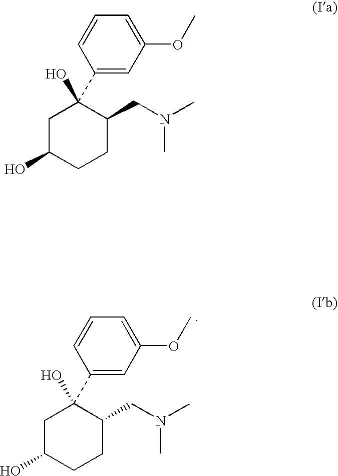

The compound 6-dimethylaminomethyl-1-(3-methoxy-phenyl)cyclohexane-1,3-diol of formula (I), its stereoisomers and corresponding salts thereof such as the hydrochloride salt as well as methods for their preparation are well known, for example, from US RE37,355 E, the entire disclosure of which is incorporated herein by reference. The compound (1RS,3RS,6RS)-6-dimethylaminomethyl-1-(3-methoxy-phenyl)-cyclohexane-1,3-diol is also known as axomadol (WHO Drug Information, Vol. 17, No. 2, 2003, List 49).

If any of the components, particularly component (a), is present as mixture of enantiomers, such a mixture may contain the enantiomers in racemic or non-racemic form. A non-racemic form could, for example, could contain the enantiomers in a ratio of 60±5:40±5; 70±5:30±5; 80±5:20±5 or 90±5:10±5.

The term “isolated” as used herein with respect to a stereoisomer means that the stereoisomer (e.g., enantiomer or diastereomer) is separated from other stereoisomers, but not necessarily from other substances.

The compound 6-dimethylaminomethyl-1-(3-methoxy-phenyl)-cyclohexane-1,3-diol, in particular the (1RS,3RS,6RS)-stereoisomer, according to component (a) may be present in the inventive combination in form of a salt, preferably an acid addition salt, whereby any suitable acid capable of forming such an addition salt may be used.

The conversion of the 6-dimethylaminomethyl-1-(3-methoxy-phenyl)-cyclohexane-1,3-diol compound, particularly the (1RS,3RS,6RS)-stereoisomer, into a corresponding addition salt via reaction with a suitable acid may be effected in a manner known to persons skilled in the art. Suitable acids include, but are not limited to, hydrochloric acid, hydrobromic acid, sulfuric acid, methanesulfonic acid, formic acid, acetic acid, oxalic acid, succinic acid, tartaric acid, mandelic acid, fumaric acid, maleic acid, lactic acid, citric acid, glutamic acid, aspartic acid, 1,1-Dioxo-1,2-dihydro-1λ⁶-benzo[d]isothiazol-3-on (saccharin), monomethylsebacic acid, 5-oxo-proline, hexane-1-sulfonic acid, nicotinic acid, 2-, 3- or 4-amino benzoic acid, 2,4,6-trimethyl-benzoic acid, α-lipoic acid, acetyl glycine, and hippuric acid. Salt formation is preferably effected in a solvent, for example diethyl ether, diisopropyl ether, alkyl acetates, acetone and/or 2-butanone.

Certain salts of 6-dimethylaminomethyl-1-(3-methoxy-phenyl)-cyclohexane-1,3-diol, in particular of the (1RS,3RS,6RS)-stereoisomer, may be preferred such as the hydrochloride salt or the salts of phosphoric acid as well as polymorphs thereof. The salts of phosphoric acid and their respective polymorphs, are disclosed, for example, in US 2006/0211887 A1, the entire disclosure of which is incorporated herein by reference.

Phosphoric acids that may be preferred are oxo acids of phosphorus. The di-(also pyro-) and the condensed meta- and polyphosphoric acids, which are also included according to the present invention can be derived from orthophosphoric acid (relative molar mass 98.0 g/mole). Primary, secondary and tertiary phosphates, which are also included according to the present invention, can be formed by stepwise replacement of the H atoms of orthophosphoric acid. Phosphate salts as also included by the present invention are understood as meaning salts from the reaction of 6-dimethylaminomethyl-1-(3-methoxy-phenyl)-cyclohexane-1,3-diol of formula I in particular with condensed phosphoric acids, such as meta- and diphosphoric acid, as well as salts of orthophosphoric acid. Salts of diphosphoric acid and orthophosphoric acid are preferred. Salts of orthophosphoric acid are particularly preferred.

It is known to persons skilled in the art that the analgesic action of NSAIDs is due to the inhibition of the enzymatic production of prostaglandins, wherein Cyclooxygenase (COX) is the key enzyme in the conversion of arachidonic acid derived from lipids of the cell membrane to prostaglandins and other eicosanoids. COX exists in two different isoforms characterized by different expression patterns. COX-I is constitutively expressed in many cells of the body and responsible mainly for the production of eicosanoids serving normal physiological functions. COX-II expression is induced during inflammation and also COX-II is expressed in the central nervous system.

Paracetamol, which is also known as acetaminophen, and its derivatives do not show any significant anti-inflammatory activity and are accordingly not considered to be NSAIDs (i.e COX-I/COX-II inhibitors). The term paracetamol and its derivatives as used herein includes said compounds in any possible form, thereby particularly including solvates and polymorphs thereof.

The term derivative as used herein particularly includes prodrugs such as ethers and esters of Paracetamol. Suitable methods for selecting and preparing a pro-drug of a given substance are, for example, described in “Textbook of Drug Design and Discovery, 3^{rd} edition, 2002, chapter 14, pages 410-458, Editors: Krogsgaard-Larsen et al., Taylor and Francis. The respective parts of said literature description are incorporated by reference and form part of the present disclosure.

Paracetamol and its derivatives such as Propacetamol and Phenidine as well as processes for their preparation are well known in the art, for example from E. Friderichs et al. “Analgesics and Antipyretics”, Ullmann's Encyclopedia of Industrial Chemistry, Sixth Edition, Wiley-VCH Verlag GmbH, Germany 2000, pages 1-22 and H. Buschmann, T. Christoph, E. Friderichs, C. Maul, B. Sundermann, “Analgesics From Chemistry and Pharmacology to Clinical Application”, 2002, Part II, Wiley-VCH Verlag, Germany. The respective parts of said literature descriptions are incorporated by reference and form part of the present disclosure.

In one embodiment of the inventive combination the derivative of Paracetamol according to component (b) is selected from the group consisting of Propacetamol and Phenidine.

A specific embodiment of the present invention is a combination comprising (a) (1RS,3RS,6RS)-6-dimethylaminomethyl-1-(3-methoxy-phenyl)-cyclohexane-1,3-diol, or a salt thereof, whereby the salt may preferably be the hydrochloride salt or the salt of a phosphoric acid, and (b) Paracetamol.

Both components (a) and (b) as part of the inventive combination may be administered in their usual daily dosage. The daily dosage of paracetamol should preferably not exceed 4 g for adults. For infants and children the daily dosage should preferably not exceed 90 mg/kg. Preferably the compound 6-dimethylaminomethyl-1-(3-methoxy-phenyl)-cyclohexane-1,3-diol such as the (1RS,3RS,6RS)-stereoisomer may be administered to a patient in a daily dosage of 1 to 1200 mg, particularly preferably in a dosage of 5 to 900 mg.

In another embodiment of the present invention the inventive combination may contain components (a) and (b) essentially in an equieffective ratio.

In yet a further embodiment of the inventive combination components (a) and (b) are present in such a weight ratio that the resulting composition will exert a synergistic effect upon administration to a patient. Suitable weight ratios can be determined by methods well known to those skilled in the art.

Both components (a) and (b) may also be present in the inventive combination in ratios deviating from the equieffective ratio. For, example, each of the components could be present in a range from 1/50 of the equieffective amount to 50 times the equieffective amount, from 1/20 of the equieffective amount to 20 times the equieffective amount, from 1/10 of the equieffective amount to 10 times the equieffective amount, from ⅕ of the equieffective amount to 5 times the equieffective amount, from ¼ of the equieffective amount to 4 times the equieffective amount, from ⅓ of the equieffective amount to 3 times the equieffective amount, or from ½ of the equieffective amount to 2 times the equieffective amount.

In another embodiment of the present invention the components (a) and (b) can be administered in a specific dosage regimen to treat one or more disorders selected from the group consisting of ostheoarthritis and pain, e.g. inflammatory pain, neuropathic pain, acute pain, chronic pain, visceral pain, migraine pain or cancer pain. Components (a) and (b) may be administered simultaneously or sequentially to one another, in each case via the same or different administration pathways. Another aspect of the present invention is therefore a method of treating one or more of ostheoarthritis and pain, e.g. inflammatory pain, neuropathic pain, acute pain, chronic pain, visceral pain, migraine pain or cancer pain, characterized in that components (a) and (b) are administered simultaneously or sequentially to a mammal, wherein component (a) may be administered before or after component (b) and wherein components (a) or (b) are administered to the mammal either via the same or a different pathway of administration. Suitable pathways of administrations include but are not limited to oral, intravenous, transdermal, intrathecal, intramuscular, intranasal, transmucosal, intraperitoneal, subcutaneous, or rectal administration. A suitable embodiment would thus be a kit in which the components of the combination of the invention, although spatially separated, are provided in a common presentation form.

The inventive combinations are toxicologically safe and are therefore suitable for treating mammals, particularly humans, including infants, children and adults.

Thus, in a further aspect the present invention relates to a pharmaceutical composition comprising an inventive combination as described herein and optionally one or more auxiliary agents. In a further aspect the present invention relates to a pharmaceutical dosage form comprising an inventive combination as described herein and one or more auxiliary agents. In one embodiment the inventive pharmaceutical dosage form additionally comprises caffeine.

In one embodiment, the inventive pharmaceutical dosage form is suitable for being administered orally, intravenously, intraperitoneally, transdermally, intrathecally, intramuscularly, intranasally, transmucosally, subcutaneously, or rectally.

The inventive formulations and dosage forms may contain auxiliary agents, for example, carriers, fillers, solvents, diluents, colorants and/or binders. The selection of auxiliary agents and of the amounts of the same to be used depends, for example, on how the drug is to be administered.

Suitable auxiliary agents in the context of this invention include any substances known to persons skilled in the art to be useful for the preparation of galenical formulations. Examples of suitable auxiliary agents include, but are not limited to, water, ethanol, 2-propanol, glycerol, ethylene glycol, propylene glycol, polyethylene glycol, polypropylene glycol, glucose, fructose, lactose, saccharose, dextrose, molasses, starch, modified starch, gelatine, sorbitol, inositol, mannitol, microcrystalline cellulose, methyl cellulose, carboxymethyl cellulose, cellulose acetate, shellac, cetyl alcohol, polyvinyl pyrrolidone, paraffins, waxes, natural and synthetic gums, acacia gum, alginates, dextran, saturated and unsaturated fatty acids, stearic acid, magnesium stearate, zinc stearate, glycerol stearate, sodium lauryl sulfate, edible oils, sesame oil, coconut oil, peanut oil, soybean oil, lecithin, sodium lactate, polyoxyethylene and polypropylene fatty acid ester, sorbitan fatty acid ester, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talcum, kaolin, pectin, crosspovidone, agar and bentonite.

Pharmaceutical formulations (dosage forms) in the form of tablets, effervescent tablets, chewing tablets, dragees, capsules, drops, juices or syrups are, for example, suitable for oral administration. Oral pharmaceutical formulations may also be in the form of multiparticulates such as granules, pellets, spheres, crystals and the like, optionally compressed into a tablet, filled into a capsule, filled into a sachet or suspended in a suitable liquid medium. Suitable oral pharmaceutical formulations may also be equipped with an enteric coating.

Pharmaceutical formulations that are suitable for parenteral, topical and inhalative administration include but are not limited to solutions, suspensions, easily reconstitutable dry preparations and sprays.

Suppositories are a suitable pharmaceutical formulation for rectal administration. Formulations in a deposit, in dissolved form, for example, in a patch optionally with the addition of agents to promote skin penetration, are examples of suitable formulations for percutaneous administration.

One or both of the components (a) and (b) may be present in the inventive pharmaceutical combination/formulation at least partially in controlled-release form. Moreover, any controlled release/immediate release combination of said components may also be present in the inventive pharmaceutical formulation. For example, one or both of the components may be released from the inventive formulation with a certain delay, e.g. if administered orally or rectally. Such formulations are particularly useful for “once-daily” or “twice-daily” preparations, which only have to be taken once a day, respectively, twice a day. Suitable controlled-release materials are well known to persons skilled in the art, e.g. from US2006/0121113 A1, the entire disclosure of which is incorporated herein by reference.

The inventive pharmaceutical formulations may be produced using materials, means, devices and processes that are well known in the prior art of pharmaceutical formulations, as described for example in “Remington's Pharmaceutical Sciences”, A. R. Gennaro (ed.), 17^{th} edition, Mack Publishing Company, Easton, Pa. (1985), in particular in part 8, chapters 76 to 93.

In order to obtain a solid pharmaceutical formulation such as a tablet, pill or capsule for example, the components of the pharmaceutical composition may be granulated with a pharmaceutical carrier, for example conventional tablet ingredients such as corn starch, lactose, saccharose, sorbitol, talcum, magnesium stearate, dicalcium phosphate or pharmaceutically acceptable gums, and pharmaceutical diluents, for example water, in order to form a solid composition that contains the components in homogeneous distribution. The term “homogeneous distribution” is taken to mean that the components are distributed uniformly over the entire composition, so that said composition may easily be divided into equally effective unit dose forms, such as tablets, pills or capsules. The solid composition is then divided into unit dose forms. The tablets or pills of the pharmaceutical composition according to the invention may also be coated or compounded in a different manner, in order to provide a dose form with a controlled release.

The amount of the inventive pharmaceutically active combination to be administered to the patient may vary depending on different factors well known to those skilled in the art, for example, the weight of the patient, the route of administration, the severity of the illness and the like.

In a further aspect the present invention relates to the use of an inventive combination as described herein as described herein for the treatment of one or more disorders selected from the group consisting of ostheoarthritis and pain.

In another aspect the present invention relates to the use of an inventive combination as described herein for the preparation of a medicament for the treatment of one or more disorders selected from the group consisting of ostheoarthritis and pain.

In yet another aspect the present invention relates to a method of treating one or more of ostheoarthritis and pain in a mammal, preferably a human, which comprises administering an effective amount of an inventive combination as described herein to the mammal.

The term pain as used herein preferably includes but is not limited to inflammatory pain, neuropathic pain, acute pain, chronic pain, visceral pain, migraine pain and cancer pain.

### Pharmacological Methods:

### A. Randall-Selitto Test in Rats

The weight ratios of the components (a) and (b) that will lead to a supra-additive effect (synergistic effect) of the inventive pharmaceutical composition may be determined via the test of Randall and Selitto as described in Arch. Int. Pharmacodyn., 1957, 111: 409 to 419, which is a model for inflammatory pain. The respective part of the literature is hereby incorporated by reference and forms part of the present disclosure.

Acute inflammation is induced by an intraplantar injection of 0.1 ml of a carrageenan solution (0.5% in distilled water) into one hind paw. The mechanical nociceptive threshold is measured 4 hours after carrageenan injection using an Algesiometer (Ugo Basile, Italy). The device generates a mechanical force with a linear increase over time. The force is applied to the dorsal surface of the inflamed rat hind paw via a cone-shaped stylus with a rounded tip (2 mm tip diameter). The nociceptive threshold is defined as the force (in grams) at which the rat vocalises (cut-off force 250 g). The mechanical nociceptive threshold is measured at different timepoints after the substance/substance combination or vehicle administration. The antinociceptive and antihyperalgesic activity of the tested substance is expressed as percentages of the maximal possible effect (% MPE). The group size is n=10.

The analysis of the results with respect to a supra-additive effect of the inventive pharmaceutical composition comprising the components (a) and (b) is carried out via statistical comparison of the theoretical additive ED50-value with the experimentally determined ED₅₀-value of a so-called fixed ratio combination (isobolographic analysis according to Tallarida J T, Porreca F, and Cowan A. Statistical analysis of drug-drug and site-site interactions with isobolograms. Life Sci 1989; 45: 947-961).

The interactions studies presented herein were performed using equieffective doses of the two components, calculated from the ratio of the respective ED₅₀ values of the components if administered alone.

The route of administration was intravenous (i.v.) for (1RS,3RS,6RS)-6-dimethylaminomethyl-1-(3-methoxy-phenyl)-cyclohexane-1,3-diol hydrochloride (A) and intraperitoneal (i.p.) for paracetamol. When A was applied alone, the peak effect was reached 15 min p. appl. (timepoint of first measurement) and ED₅₀-value of 15.80 (14.46-17.36) mg/kg i.v. was calculated. Paracetamol induced a dose-dependent analgesic effect with ED₅₀-values of 189.9 (181.3-198.4) mg/kg i.p., reaching the peak effect 120 min post administration. According to their respective timepoint of peak effect, A was applied 15 min and paracetamol 120 min before timepoint of measurement of the interaction-experiments (i.e. paracetamol was administered 105 min before A). Thus, the time point of ED₅₀ calculation of the combination corresponds to the timepoint of the peak effect of the respective compound. The isobolographic analysis revealed that the experimental ED₅₀-values of the combinations were significantly lower than the respective theoretical ED₅₀-values. Thus, the combination studies demonstrate significant synergistic interaction of A with paracetamol.

The results of the isobolographic analysis are summarized in the following Table 1.

TABLE 1
Experimental ED₅₀ values of A and Paracetamol and isobolographic
analysis of the interaction between A and Paracetamol:

| Substance/ | | | Theoretical | Experimental | |
|---|---|---|---|---|---|
| ED₅₀ | | | ED₅₀ of the | ED₅₀ of | |
| [mg/kg] | A | Paracetamol | combination | combination | Interaction |
| A+ | 15.80 | 189.9 | 102.86 | 80.71 | supra-additive |
| Paracetamol | (14.46-17.36) | (181.3-198.4) | (97.68.-108.04) | (67.94-90.62) | (p < 0.001) |

p: level of statistical significance

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A composition of matter comprising in combination:
(a) a 6-dimethylaminomethyl-1-(3-methoxy-phenyl)cyclohexane-1,3-diol compound, and
(b) paracetamol or a derivative thereof.

2. A composition of matter as claimed in claim 1, wherein said 6-dimethylaminomethyl-1-(3-methoxy-phenyl)cyclohexane-1,3-diol compound is 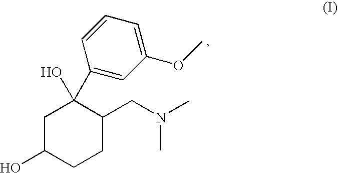
or a pharmaceutically acceptable salt thereof.

3. A composition of matter as claimed in claim 1, wherein said 6-dimethylaminomethyl-1-(3-methoxy-phenyl)cyclohexane-1,3-diol compound is in the form of an isolated stereoisomer.

4. A composition of matter as claimed in claim 1, wherein said 6-dimethylaminomethyl-1-(3-methoxy-phenyl)cyclohexane-1,3-diol compound is in the form of a mixture of stereoisomers in any mixing ratio.

5. A composition of matter as claimed in claim 4, wherein said mixture is a racemic mixture.

6. A composition of matter as claimed in claim 1, wherein said 6-dimethylaminomethyl-1-(3-methoxy-phenyl)cyclohexane-1,3-diol compound is (1RS,3RS,6RS)-6-dimethylaminomethyl-1-(3-methoxy-phenyl)-cyclohexane-1,3-diol or a salt thereof.

7. A composition of matter as claimed in claim 6, wherein said 6-dimethylaminomethyl-1-(3-methoxy-phenyl)cyclohexane-1,3-diol compound is a hydrochloride salt or a salt of phosphoric acid.

8. A composition of matter as claimed in claim 1, comprising a derivative of paracetamol selected from the group consisting of propacetamol and phenidine.

9. A composition of matter as claimed in claim 1, wherein the 6-dimethylaminomethyl-1-(3-methoxy-phenyl)-cyclohexane-1,3-diol compound and the paracetamol or derivative thereof are present in a weight ratio such that the composition exerts a synergistic effect upon administration to a patient.

10. A composition of matter as claimed in claim 1, in the form of a pharmaceutical dosage form suitable for oral, intravenous, intraperitoneal, intradermal, intrathecal, intramuscular, intranasal, transmucosal, subcutaneous, or rectal administration.

11. A composition of matter as claimed in claim 1, wherein the 6-dimethylaminomethyl-1-(3-methoxy-phenyl)cyclohexane-1,3-diol compound or the paracetamol or derivative thereof, or both, is present in controlled-release form.

12. A method of treating a condition selected from the group consisting of osteoarthritis and pain in a subject, said method comprising administering to said subject a pharmacologically effective amount of a composition of matter as claimed in claim 1.

13. A method as claimed in claim 12, wherein said condition is pain is selected from the group consisting of inflammatory pain, neuropathic pain, acute pain, chronic pain, visceral pain, migraine pain and cancer pain.

14. A method as claimed in claim 12, wherein the 6-dimethylaminomethyl-1-(3-methoxy-phenyl)-cyclohexane-1,3-diol compound and the paracetamol or derivative thereof are administered simultaneously to said subject.

15. A method as claimed in claim 12, wherein the 6-dimethylaminomethyl-1-(3-methoxy-phenyl)-cyclohexane-1,3-diol compound and the paracetamol or derivative thereof are administered by the same route of administration.

16. A method as claimed in claim 12, wherein the 6-dimethylaminomethyl-1-(3-methoxy-phenyl)-cyclohexane-1,3-diol compound and the paracetamol or derivative thereof are administered by different routes of administration.

17. A method as claimed in claim 12, wherein the 6-dimethylaminomethyl-1-(3-methoxy-phenyl)-cyclohexane-1,3-diol compound and the paracetamol or derivative thereof are administered sequentially to said subject.

18. A method as claimed in claim 17, wherein the 6-dimethylaminomethyl-1-(3-methoxy-phenyl)-cyclohexane-1,3-diol compound is administered before the paracetamol or derivative thereof.

19. A method as claimed in claim 17, wherein the 6-dimethylaminomethyl-1-(3-methoxy-phenyl)-cyclohexane-1,3-diol compound is administered after the paracetamol or derivative thereof.
